(19) United States
(12) Patent Application Publication
(10) Pub. No.: US 2021/0368853 A1
(43) Pub. Date: Dec. 2, 2021

(54) Nicotine Aerosol Formulation

(71) Applicant: Andrus Research Inc., Ancaster (CA)

(72) Inventors: Paul Andrus, Ancaster (CA); Gayle Campbell-Andrus, Ancaster (CA); Michael Bielejewski, Hamilton (CA)

(21) Appl. No.: 17/395,876

(22) Filed: Aug. 6, 2021

Related U.S. Application Data
(63) Continuation-in-part of application No. PCT/CA2020/050150, filed on Feb. 6, 2020.
(60) Provisional application No. 62/886,033, filed on Aug. 13, 2019.
(60) Provisional application No. 62/802,781, filed on Feb. 8, 2019.

(51) Int. Cl. A24B 15/167; A24F 40/42; A24F 40/10

(52) U.S. Cl. CPC A24B 15/167; A24F 40/10; A24F 40/42

(57) ABSTRACT

An orally inhalable formulation is provided comprising nicotine or a pharmaceutically acceptable salt thereof, a monohydric alcohol, menthol or a menthol-containing essential oil and a propellant. The formulation may be provided in a pressurized metered dose inhaler.

### FIELD OF THE INVENTION

The present invention relates to inhalable formulations, and in particular, to novel inhalable formulations comprising nicotine for use in a standard pressurized metered dose inhaler for the purpose of tobacco cessation or tobacco harm reduction.

### BACKGROUND OF THE INVENTION

There remains an unmet need within the nicotine “vaping” industry to provide an aerosol with a more optimal set of physical characteristics which would allow provision of a very satisfying and immediate nicotine “hit” while avoiding excessive throat irritation and aversiveness including nausea at higher doses. Electronic inhalers or “vaporizers” have the inherent weakness in this regard of requiring heat to generate the aerosol which tends to cause volatile nicotine to quickly enter the gas phase from the liquid droplet phase, and thereby result in much of the nicotine dose being absorbed onto the throat and upper airway through which it passes too slowly into the bloodstream to allow efficient brain delivery. A larger oropharyngeal fraction also causes nausea.

Recent particle size examination of e-cigarette aerosol has shown droplet sizing in the 0.5 micron range very comparable to that of cigarette smoke (Mulder et.al., Scientific Reports (2019) 9:10221). This would suggest that the e-cigarette should closely mimick the effect of the tobacco cigarette. However most experienced smokers who smoke primarily for nicotine stimulant effect on an hourly basis find that the e-cigarette experience bears little resemblance to that of a tobacco cigarette in this regard, and is completely inadequate as a sustainable nicotine delivery system. This conundrum can be reconciled. Although of identical “particle” size, tobacco smoke is comprised of nicotine-containing solid particles, and e-cigarette aerosol contains liquid droplets of a propylene glycol solution of nicotine. This difference has a profound effect on the ratio of upper to lower airway drug deposition. In a particle sizing examination, e-cigarette aerosol in drawn through a cascade impactor. The propylene glycol and the nicotine residue deposited (and remaining) on the glass throat and the impactor stages is then weighed to determine the size distribution. While a fraction of the nicotine remains within fine droplets and is available for deep lung absortion, the sizing only indicates the distribution of the nicotine which did not yet vaporize. What is not accounted for is the degree of vaporization of the heated volatile liquid nicotine from the propylene glycol into the gas phase. That the nicotine and its solvent are quickly separating is suggested since no propylene glycol residue is found on the throat component of the sizing apparatus despite significant nicotine residue found there. The variability of the throat component nicotine residue may relate to variation in aerosol temperature with voltage, and further vaporization of the nicotine from the throat component with airflow through the apparatus thereby escaping measurement. Unlike an aerosol sizing apparatus, the moist mucous membrane of the human upper airway very efficiently collects hygroscopic nicotine vapour. So, although e-cigarette aerosol contains a fine propylene glycol droplet fraction with some residual nicotine available for deep lung penetration, what is most important from a smoking experience standpoint is the ratio of upper to lower airway nicotine deposition. Too much upper airway deposition causes nausea before there is adequate lower airway deposition to create effective central nervous system stimulation.

By contrast, standard respiratory medicine delivery devices such as pressurized metered dose inhalers have been designed to efficiently deposit a majority of the drug to the lung through which nicotine would be rapidly absorbed via the pulmonary circulation and efficiently delivered to the brain as occurs with cigarette smoking. While the standard metered dose inhaler is very cost effective and drug delivery efficient, it discharges the aerosol too quickly to avoid a significant minority fraction of the aerosol from depositing in the throat. This weakness is particularly evident and dose limiting in the case of a harsh alkaloid such as nicotine, which is combined with a solvent such as ethanol that may also be an irritant within the throat. Consequently, traditional drug solution formulation strategies for metered dose inhalers in general are not optimal in the unique case of nicotine for which they were not originally intended.

Since the late 1990's, medicinal pressurized metered dose inhalers have typically comprised a solution formulation of a respiratory drug dissolved in a co-solvent such as ethanol, which is then dissolved in hydrofluoroalkane propellant under pressure within an aluminum canister. This industry formulation shift away from solid drug particle suspension formulations was coupled with new fine-bore nozzle technology which together for the first time allowed the generation of a much finer aesosol that could deposit a majority of the drug dose into the lungs. Prior to this, a metered dose inhaler (MDI) could at best delivery only a minority of the drug into the lungs with the majority landing in the mouth and oropharynx. In order to ensure sustained drug solution and stability over time, and avoid precipitation and then plugging of the fine bore nozzle, typically the respiratory drug would be present at about 10% of the weight of the ethanol co-solvent. A particle sizing study of a 10% solution formulation of nicotine in ethanol with fine bore nozzle (0.3 mm) demonstrated that it was possible to generate a nicotine aerosol from a metered dose inhaler with a sufficiently fine particle size to allow the majority of the nicotine to be delivered to the lungs as with tobacco smoke (Andrus et. al. Can. Respir. J. 1999). In the case of an asthma drug such as salbutamol, a significant minority fraction of the dose being deposited in the throat and upper airway is not actually undesirable (as it is with nicotine) because salbutamol is not harsh, and asthma affects both upper and lower airways. However, in the case of nicotine, even a minority fraction of the nicotine dose landing in the throat is very dose limiting.

Another problem in the practical use of an MDI for nicotine delivery is the need to concentrate the nicotine on a per puff basis to a degree that allows deep delivery comparable to a cigarette puff while avoiding significant upper airway deposition and harshness. Menthol is widely used in cigarettes and can be added to an MDI formulation to decrease the harshness of nicotine. However, menthol is crystalline at room temperature and therefore requires an alcohol solvent to create a liquid for a solution formulation. Pure menthol (or “methol” as an alcohol-based liquid) has a tolerable but unpleasant taste characteristic.

Nicotine-containing aerosol formulations have previously been described. Gildemeister (GB 1,528,391) discloses nicotine in ethanol solution formulations including one having a very low ethanol:nicotine ratio of 1.5:1, and one having a very high ethanol:nicotine ratio of 250:1. The low ethanol example also contains “methol” which is described as an essential oil presumeably high in menthol at 4.5 times the level of nicotine. Menthol is an effective counter-irritant to the irritant effect of nicotine, however when in gross excess to nicotine it becomes itself a barrier to smooth aerosol inhalation. This excess menthol irritant effect becomes more apparent over time with sustained use.

More recently, Lechuga-Ballesteros (US 2006/0018840 A1) teaches that lowering the ethanol co-solvent to as low as 1% of the total formulation weight does provide benefit to the fine particle fraction. However, the formulation examples given in US 2006/0018840 A1 essentially adhere to the standard 10% nicotine in ethanol solution and the examination of lowering the ethanol to 1% in FIG. 3 of this example implies a lower nicotine dose per puff with low ethanol while maintaining the standard 10:1 ethanol:nicotine ratio, not a high nicotine quantity concentrated in a low ethanol quantity. This reference also teaches that a neutralizing acid such as lactate should ideally be provided in molar excess (1.2:1) such that no residual free base remains.

Hearn (US 2015/0374034 A1) discloses multiple formulation examples, all of which more or less conform to the standard 10:1 ethanol:nicotine ratio. A representative formulation is listed on page 9 and contains 0.95% ethanol and 0.056% nicotine or a 17:1 ratio. This citation does warn of difficulties in maintaining nicotine solubility in co-solvent alcohols and suggests on page 5 that “Nicotine salts may also be in liposomal encapsulation. Such encapsulation may allow the nicotine concentration of a formulation to be further increased without nicotine precipitation occurring”. This implies that it would not be advisable to increase the risk of having nicotine precipitate from solution by lowering ethanol and other alcohol amounts substantially relative to nicotine.

Thus, it would be desirable to provide an improved aerosol formulation for use in medicinal pressurized metered dose inhaler (pMDI) for the delivery of nicotine.

### SUMMARY OF THE INVENTION

Nicotine aerosol formulations have been designed for use with standard medicinal pressurized metered-dose inhalers.

In a first aspect, an orally inhalable nicotine formulation is provided comprising nicotine or a pharmaceutically acceptable salt thereof, a monohydric alcohol, menthol-containing essential oil and a propellant, wherein the ratio of monohydric alcohol:nicotine by weight is no more than about 2.5:1, and the ratio of menthol-containing essential oil/nicotine is about 0.25-2.0:1 by weight. This formulation provides enhanced delivery of nicotine to the lungs via an inhaler, particularly an inhaler not having a holding chamber.

The resulting formulation possesses physical properties, such as viscosity and volatility, which enhance its use with a standard pressurized metered-dose inhaler not having a holding chamber, and provides an ultrafine, low volume aerosol with an improved fine particle fraction without loss of nicotine solubility, and results in less throat irritation relative to deep lung nicotine delivery.

While not wishing to be bound by any particular theory, the formulation strategy in this aspect is to use menthol-containing essential oil as a nicotine cosolvent to facilitate very low ethanol/alcohol cosolvent content without compromising nicotine solubility. The net result of this strategy is to create a formulation with properties including viscosity and nicotine volatility that results in an aerosol having a large fine particle fraction, and a volatility that decreases nicotine evaporation and thereby reduces nicotine delivery in the upper airway as occurs with heat vaporizers. This results in greater deep lung nicotine delivery relative to upper airway delivery, as well as greater drug exhalation. Such a deviation from standard formulating practice would be undesirable in the case of a respiratory medicine, but has advantages in the particular case of nicotine since the desired target is almost exclusively the deep lung, and for which a degree of drug wastage is less important. The overall effect of lowering and stabilizing the aerosol particle size in this manner is to improve the ratio of deep lung (brain) delivery relative to throat harshness, whatever the nominal dose discharged from the inhaler.

In another aspect, an orally inhalable nicotine formulation is provided comprising nicotine, or a pharmaceutically acceptable salt thereof, a monohydric alcohol in an amount of 6-12% by wt of the formulation, lactic acid in an amount of 0.4-0.6:1 nicotine by wt, menthol in an amount of 1-2:1 nicotine by wt and a propellant, which provides enhanced delivery of nicotine to the lungs via an inhaler having a holding chamber.

Thus, the present invention employs two divergent formulation approaches, one primarilty for use with a pMDI in the absence of a holding chamber where an ultrafine and low volume aerosol is desirable, and one for use with a pMDI having an aerosol holding chamber where aerosol stability is desirable. The inventors have recognized that the relative balance of ingredients within nicotine aerosol formulations is important to successfully reproduce the psychoactive effect of a cigarette over time in a satisfying and tolerable manner.

### BRIEF DESCRIPTION OF THE FIGURE

FIG. 1 illustrates an exemplary metered-dose inhaler that may be used to administer the present nicotine formulation.

### DETAILED DESCRIPTION

In a first aspect, an orally inhalable nicotine formulation is provided comprising nicotine or a pharmaceutically acceptable salt thereof, dissolved in a combination of a monohydric alcohol and menthol-containing essential oil as co-solvents, and a propellant, wherein the ratio of monohydric alcohol to nicotine is no more than about 2.5:1 w/w, and the ratio of menthol-containing essential oil:nicotine by weight in is the range 0.25-2.0:1 w/w. This formulation is useful with a pMDI which does not have a holding chamber.

The nicotine in the present formulations may be in free base form, or in the form of a pharmaceutically acceptable salt. Preferably, the nicotine is in the form of an acid salt formed by conjugation with an appropriate conjugate acid having a sufficiently low pKa to maintain the nicotine base in solution. Examples of suitable conjugate acids include, but are not limited to, lactic acid, salicylic acid, sorbic acid, benzoic acid, pyruvic acid, lauric acid, and levulinic acid. In a preferred embodiment, nicotine in the form of a lactic acid salt is used in the present formulation.

Alternatively, nicotine in free base form is combined with an appropriate conjugate acid and the selected monohydric alcohol and menthol-containing essential oil to result in formation of the nicotine acid salt in situ. In this first aspect, the conjugate acid is added to the formulation at about a 0.8:1 molar equivalent level relative to the nicotine content.

The nicotine free base or acid salt is dissolved in a monohydric alcohol and menthol-containing essential oil. A preferred monohydric alcohol is ethanol. To achieve the advantages of this aspect of the present invention, the ratio of monohydric alcohol to nicotine is no more than about 2.5:1 w/w, preferably less than 2:1 w/w, and more preferably about 1.5:1 w/w, or 1.2:1 w/w. The ratio of menthol-containing essential oil:nicotine by weight in is the range 0.25-2.0:1 w/w, preferably in the range of 0.5-1.5:1 w/w, such as 0.5-1.2:1 w/w menthol-containing essential oil:nicotine or 1:1 w/w menthol-containing essential oil:nicotine. More preferably, the ratio of menthol-containing essential oil:nicotine by weight is in the range of 0.5-0.9:1 w/w, or 0.7:1. As one of skill in the art will appreciate, the amount of menthol-containing essential oil will vary with the amount of menthol in the selected essential oil.

The nicotine/alcohol/menthol-containing essential oil mixture is then dissolved in a suitable propellant, e.g. a hydrofluoro-based propellant. Examples of suitable hydrofluoroalkane propellants include, for example, HFA 334a, 134a, 152a, 227ea, etc. Hydrofluoroolefin propellants (HFO's) may also be used. One example is HFO 1234ze.

The present formulations will include a standard amount of the hydrofluoroalkane propellant, for example, an amount in the range of about 12-18 ga (e.g. 12-15 g) per 19 mL canister. As one of skill in the art will appreciate, the nicotine concentration in the propellant will vary with the inhaler used to deliver the formulation, and the desired dose of nicotine per actuation. For example, to achieve delivery of 1 mg nicotine per actuation, a concentration of about a 1.6% nicotine in a propellant solution using a standard 50 microliter metering valve inhaler, while a greater nicotine concentration is required if an inhaler with a 25 microliter metering valve is used, for example, a 3.6% nicotine in propellant. The concentration of nicotine in propellant would be decreased or increased to achieve delivery of less or more nicotine per actuation, accordingly.

The menthol-containing essential oil is an essential oil extract containing high levels of (−)menthol, e.g. greater than 20% by wt, and preferably greater than 25%, 30% or 40% by wt. The menthol-containing essential oil may be obtained from the Mentha genus of plants, including but not limited to, watermint (M. aquatica), wild or corn mint (M. arvensis), Japanese mint (M. arvensis var. piperascens), and hybrid mints such as peppermint (M. aquatica x M. spicata) and Scotch spearmint (M. arvensis x M. spicata), or a combination thereof. The menthol-containing essential oil is obtained using well-established techniques, for example, extraction from dried or fresh leaves and the flowering tops of the plant using alcohol. Such menthol-containing essential oils are also readily commercially available. The flavour of the formulation will vary with the menthol-containing oil used. To alter flavour, a combination of menthol-containing essential oils may be used, for example, peppermint oil combined with corn or Japanese mint.

The menthol-containing essential oil contains a large fraction of menthol (e.g. greater than 20%, 30%, 40%, 50%, 60% or more, for example, in a range of about 40-80%) and functions as a co-solvent with the monohydric alcohol, as well as providing a pleasant natural flavor. Thus, the presence of the mint oil facilitates lowering the ethanol content without compromising nicotine solubility in the context of the actuator nozzle of an inhaler, e.g. a fine bore actuator nozzle prone to plugging.

In the first aspect, the formulation comprises a preferred ethanol:nicotine ratio of 1.5:1 w/w, and a menthol-containing essential oil:nicotine ratio of 1:1. The range of nicotine doses on a per puff basis ranges from 0.07-1.2 mg.

The nicotine formulation comprising monohydric alcohol and menthol-containing essential oil as co-solvents, in which the ratio of monohydric alcohol to nicotine is no more than about 2.5:1 w/w, and the ratio of menthol-containing essential oil:nicotine is the range 0.25-2.0:1 w/w, provides an ultrafine, low volume aerosol with an improved fine particle fraction without loss of nicotine solubility, and results in less throat irritation relative to deep lung nicotine delivery via a pMDI not having a holding chamber.

In another aspect, an orally inhalable nicotine formulation is provided comprising nicotine, or a pharmaceutically acceptable salt thereof, a monohydric alcohol in an amount of 6-12% by wt of the formulation, lactic acid in an amount of 0.4-0.6:1 nicotine by wt, menthol in an amount of 1-2:1 nicotine by wt and a propellant. This formulation is particularly useful with a pMDI having a holding chamber.

This formulation is designed to enhance the psychoactive effect of the formulation, while neutralizing nicotine-induced irritation and bronchoconstriction, particularly when the formulation is used with a metered dose inhaler (MDI) having an aerosol holding chamber or “spacer”. Thus, in this formulation, an increased menthol to nicotine content (M:N content) is utilized which has surprisingly been determined to effectively offset the lower airway bronchoconstricting effect of the nicotine. The M:N in the present formulation is 1-2:1 w/w, and preferably, the M:N is 1-1.5 w/w.

In addition to neutralizing the bronchoconstricting effect of nicotine, an M:N content of 0.5-2:1 w/w, and preferably 1-1.5:1 w/w, was observed to provide another unexpected effect over time. In particular, this M:N content results in a reduction in abdominal body fat and an increase in lean muscle mass in the absence of any change in diet or exercise. While not wishing to be bound by any particular theory, menthol has been shown to be an allosteric modulator of GABA receptors in the brain, and when deposited on the lung and delivered to the brain, such GABA-ergic substances are known to transiently increase growth hormone, such as testosterone in men, similar to that which occurs with exercise, which may then account for the body fat reduction and increased lean muscle mass. Further, while delivery of nicotine and menthol in isolation can be intolerable, and delivery in ratios outside of the present range may be less effective, use in the M:N range of 1-2:1 w/w in medical-grade aerosol form appear to act synergistically to provide a positive physical impact.

Menthol for use in this formulation is in the form of menthol crystals which are easily dissolved in the ethanol content of this formulation.

Further, in view of the fact that it is the nicotine free base that is harsh and causes throat irritation, organic acid is added in an amount that results in a physiologic to basic pH that is physiologically acceptable, e.g. in the range of about 7.4-7.8. The term “physiologically acceptable” refers to a formulation having a pH that is acceptable for inhalation, without any undue adverse affect, such as excessive toxicity. For example, an organic acid (e.g. lactic acid) may be added to the formulation in an amount of 0.4-0.6:1 nicotine w/w to achieve the desired pH. In an embodiment, the organic acid is added to the formulation in a weight ratio of 0.5:1 acid:nicotine. This ratio of acid to nicotine recognizes that the nicotine and acid salt ions are dissociated in an aqueous solution as in the lungs, and provides for a physiologic to slightly basic pH in situ. This more appropriately reduces the harsh effect of the nicotine free base to achieve smooth inhalation properties in which the irritation caused by the nicotine is reduced or altogether eliminated, and also avoids an excess of free acid in the airways which is also quite irritating.

To further reduce the irritation caused by inhalation of nicotine, in this formulation the nicotine is diluted in a higher volume of monohydric alcohol co-solvent (such as ethanol) rather than the reduced volumes used conventionally. Thus, the formulation incorporates a monohydric alcohol co-solvent in a fixed amount in the range of 6-12% by weight of the formulation for all nicotine concentrations which has been found to provide a stable aerosol that exhibits much less upper airway harshness when used with an MDI having an aerosol holding chamber. A preferred range of monohydric alcohol in the formulation is about 9-10% by wt. An additional benefit of an increased volume of monohydric alcohol co-solvent is the dilution effect on all active ingredients, thereby permitting the inclusion of sufficient menthol in the formulation to counter bronchoconstriction without an excessive minty taste.

This formulation, for use in a pMDI with a chamber, may be used with any nicotine content. In embodiments, the nicotine content is in an amount in the range of 0.125-2% by wt of the formulation.

The propellant used in this formulation, for use in a pMDI with a chamber, is as described above, and incorporated in similar amounts.

The nicotine aerosol formulations of the present invention are designed for use with a standard medicinal pressurized metered-dose inhaler made up of three primary components: a canister, a metering valve and an actuator. The canister is typically formed from stainless steel or aluminum, and will contain the formulation. In a preferred embodiment, the inside surface of the aluminum canister is coated to prevent nicotine adsorption and crystallization onto its surface, for example with a plasma coating. The metering valve is sealed to the canister, and includes a movable hollow valve stem. The metering valve is configured to release a metered quantity of the formulation from the canister through the valve stem. The actuator comprises a hollow body that receives the canister, a mouthpiece, typically projecting obliquely from the body, and an actuator or atomizing nozzle that projects inwardly at the junction of the body and the mouthpiece. Formulation is administered by pressing the canister into the body of the actuator while inhaling through the mouthpiece.

The actuator may be paired with a compact dilution spacer (for example, as described in U.S. Pat. No. 10,052,445) to further facilitate deep lung delivery of the nicotine formulation, and to further reduce oropharyngeal deposition, by slowing and diluting the aerosol stream, and rendering less sensitive the timing of actuation relative to inhalation which is normally required to use a standard pMDI effectively. The aerosol plume develops within the dilution chamber before becoming entrained and redirected towards the mouthpiece by the inhalation airstream.

Embodiments of the invention are described by reference to the following examples which are not to be construed as limiting:

### EXAMPLE 1

### Nicotine Formulation

A number of variations of the present nicotine formulation was prepared using a fixed nicotine content of 0.2 mg which aligns with previously studied nicotine MDI formulations (Caldwell, B. O and Crane, J, Nicotine and Tobacco Research, 2016). This experimental example was conducted using the pMDI in the absence of a holding chamber. Starting with an ethanol:nicotine w/w ratio of 7:1 along with crystalline menthol dissolved in the alcohol at a menthol:nicotine ratio of 0.5:1, the ethanol:nicotine w/w ratio was incrementally lowered and sampled by several users using a standard inhaler. A reduced level of harshness became noticeably evident at an ethanol:nicotine ratio of 2.5:1. The lowest ratio tried was 1.25:1 which was substantially less harsh than 2.5:1. However, the nicotine hit effect was significantly attenuated at the lower end of the ethanol:nicotine ratios. Higher dose inhalers at 0.3 mg nicotine per puff were then prepared at an ethanol:nicotine ratio of 1.5:1 and these were found to recover the degree of nicotine hit of the lower nicotine (ethanol:nicotine 7:1 inhaler), without the level of harshness of the lower nicotine inhaler, a further issue arose in that plugging of the actuator nozzle occurred to a partial or complete degree sporadically and was often not recoverable with rinsing of the actuator. As noted above, crystalline menthol was dissolved in the ethanol to function as an anti-harshness agent, with some difficulty at the lower ethanol levels. The lower ethanol levels also resulted in a nicotine solubility issue as seen by plugging of the fine bore nozzle of the inhaler.

To address these issues, a novel co-solvent strategy was employed in which peppermint oil was used as both a co-solvent and a menthol carrier since a typical peppermint oil contains menthol and menthone as the major mentholoids (menthol content can vary significantly). Peppermint oil, having a menthol content of about 42% and a menthone content of about 23%, was used at various relative levels to ethanol with a ratio of peppermint oil:nicotine of about 1.0:1.0 w/w, and the ethanol:nicotine ratio at about to 1.2:1 w/w. with lactate at 0.67 w/w to nicotine (which is about 1.2:1 lactate/nicotine molar ratio. The total mixture's weight (nicotine, ethanol and peppermint oil) was raised relative to the fixed HFA propellant of 14 g per canister. In terms of nominal nicotine content per actuation, 0.25, 0.5, 0.75. 1.0, 1.25, 1.5 and 2 mg per actuation were tried.

Various menthol oil:nicotine ratios were tried, including a peppermint oil:nicotine ratio of 1.36:1 w/w and 0.96:1 w/w, and a corn mint oil:nicotine ratio of 0.7:1 w/w. It was found that the higher the level of mint oil, the stronger the mint flavour and there was some throat irritation at the highest level of oil used. Personal preference comes into play with respect to the amount of mint flavour that is considered pleasant or desirable. A menthol oil:nicotine ratio of at least about 0.5:1 w/w was found to provide desirable results. Combinations of menthol-containing essential oils were also considered. A preferred formulation contained a mint oil:nicotine ratio of 0.7 with a combination of 80% corn mint oil and 20% peppermint oil. None of the menthol oil formulations caused plugging of the metering valve.

Thus, it was found that not only was the taste dramatically improved but the same actuator and nozzle could be used with multiple refill canisters without any inhaler nozzle plugging or narrowing tendency.

Further, it was unexpectedly found that the taste/harshness did not rise in a linear fashion with nicotine concentration as nicotine concentration was raised in conjunction with an aerosol holding chamber. In fact, the 1.0 mg per actuation nicotine dose was considered optimal by multiple experienced samplers. The lower concentrations required a greater number of actuations to achieve a cigarette-equivalent effect and were no less harsh on a per actuation basis. Consequently, the concentrations below 1 mg were overall a less satisfying experience. The 1 mg nicotine inhaler would appear to be an excessive dose when compared to the total expected nicotine delivery of a cigarette of about 1-3 mg. However, a substantial portion of the nominal dose of the inhaler is lost within the dilution spacer as well as during exhalation such that the net effect on a per puff basis is very similar to a strong cigarette. When comparing the nicotine concentration in the HFA solvent to typically available e-liquid concentrations, the 1 mg per actuation nicotine dose appears reasonable as it is only 1.6% nicotine in HFA solution compared to 5% nicotine in propylene glycol e-liquids commonly used. The nicotine concentration of the present formulation could be increased to 3 mg nicotine per actuation or 4.8% nicotine in HFA to be comparable to that of commercially used e-liquid. A user can choose anywhere from one to ten puffs to achieve their individual cigarette equivalent effect. Although higher nicotine concentrations were more inhalable with the use of a holding chamber, it became apparent that no modifying factor has greater impact on smoothness and tolerability than the presence of a holding chamber, and that the low ethanol formulation did not take full advantage of this device advantage.

### EXAMPLE 2

### Nicotine Formulation with Enhanced Effects

An alternative formulation strategy was conceived, particularly designed for use exclusively with an aerosol chamber or “spacer” attached to the pMDI component. Since a large fraction of the aerosol will land within the spacer (the fraction which would tend to land undesirably in the throat and upper airway in the absence of a spacer), a different approach to the aerosol formulation was found to have several advantages.

It was found through experimenting with smokers that instead of designing for an ultrafine aerosol which is necessary in the absence of a spacer, the ethanol content is best set at a fixed and relatively high level irrespective of nicotine content for use with an inhaler having a spacer. This approach provides a constantly rich and voluminous ethanol base to dilute the nicotine and provides resistance to evaporation as the aerosol moves through the spacer and upper airways since, generally, only respirable droplets make it out of the spacer. Nicotine vapor is extremely irritating on the throat and evaporation must be delayed/avoided at least until the aerosol has reached the peripheral lungs.

Several experimenters endorsed the nicotine formulation as having increased smoothness and ease of inhalation as the base ethanol content of the formulation as a fraction of the total formulation was raised progressively from 3% to 4.5% to 6% to 9% to 12%. A clear improvement still occurred from 6% to 9% with little difference apparent from 9% to 12%. This experiment was done with nicotine set at 2% (lactate 1%) and a menthol:nicotine ratio of 1:1 (i.e. menthol 2%), which is essentially the harshest formulation within the scope of the formulation, so that the impact of ethanol dilution could best be revealed. It appeared that this impact had mostly to do with just volumizing the aerosol when used with the spacer, as doubling all ingredients including nicotine (except HFA) in a fixed proportion also was perceived as smoother to inhale. For example, 1.5% nicotine in 4.5% ethanol was counterintuitively inferior to 3% nicotine in 9% ethanol. This led to the application of a fixed volume of 9% ethanol to all lower nicotine concentrations with favorable tolerability among more sensitive longer term smokers for whom formulations comprising 0.125-0.25% nicotine is more suitable.

Menthol was provided in varying levels relative to nicotine to several experimenters. This experiment was done with nicotine at 0.25% (0.125% lactate) and ethanol 9%, as menthol masking is most critical to more sensitive smokers for whom the lowest nicotine concentrations are preferred. While menthol is relatively easy to inhale and can be provided in gross excess of nicotine when nicotine is relatively low, it was found that there is a threshold effect at a menthol:nicotine ratio of about 1.5:1. As the M:N ratio was raised above 1.5 there was little further benefit for nicotine masking, and menthol itself became more of a significant irritant with sustained use. With the M:N ratio between 0.5 and 1.5, it becomes a matter of personal preference, but clearly 1.5 was smoother and easier to inhale than 1.0, and was quite pleasant and sustainable over time. Those who were less airway sensitive preferred M:N of about 1.0. As a point of reference, menthol cigarettes have an average M:N of 0.5, however cigarettes retain the “benefit” of tar which occurs at ten times nicotine and appears to have a masking effect.

Free base nicotine is an alkaloid that is extremely harsh to inhale. Thus, lactic acid was also varied by adding lactate in slight molar excess of nicotine at a molar ratio of lactate:nicotine of 1.1:1 (weight ratio 0.67:1 of 90% lactic acid). Since physiologic pH is not neutral but slightly basic at 7.4, the amount of lactate was then reduced to result in a pH at or very slightly above 7.4 to achieve less irritation. This relative fine tuning along the steep portion of the pH titration curve (in which lactate was reduced) was endorsed as clearly smoother to inhale by several experimenters, at a lactate:nicotine molar ratio of 0.81:1 (weight ratio 0.5:1 of 90% lactic acid). For this experiment nicotine was 1.7% (1 mg per actuation using a spacer), and the menthol:nicotine ratio was low at 0.5 so as to minimize menthol masking and thereby to most reveal the impact of pH adjustment.

The most unexpected finding of all in working with test smokers was how widely ranging their familiar nicotine intake was on a per cigarette basis. Typical cigarette consumption was narrow ranging around 25-30 per day. However extremely low and extremely high tolerance for nicotine inhalation were both common. Many smokers found 0.125-0.25% (72-144 micrograms per inhaler actuation) nicotine as a percentage of the total formulation (comprised mostly of about 18 g of HFA propellant) to be not only the limit of tolerance but that 2-4 actuations entirely satisfied their nicotine cravings. Many other smokers, generally younger with less smoking history (and perhaps less chronic airway changes and thereby less sensitivity), found that 2% nicotine (1152 micrograms per actuation) was not only easy to inhale but that 2-4 puffs was in keeping with their cigarette experience. The actual nicotine quantities consumed would be less, however, than the nominal actuated dose as the spacer device collected most of what would have landed in the mouth and throat (detrimentally), and some ultrafine droplets were exhaled.

### EXAMPLE 3

### Exemplary Inhaler for Use with the Nicotine Formulation

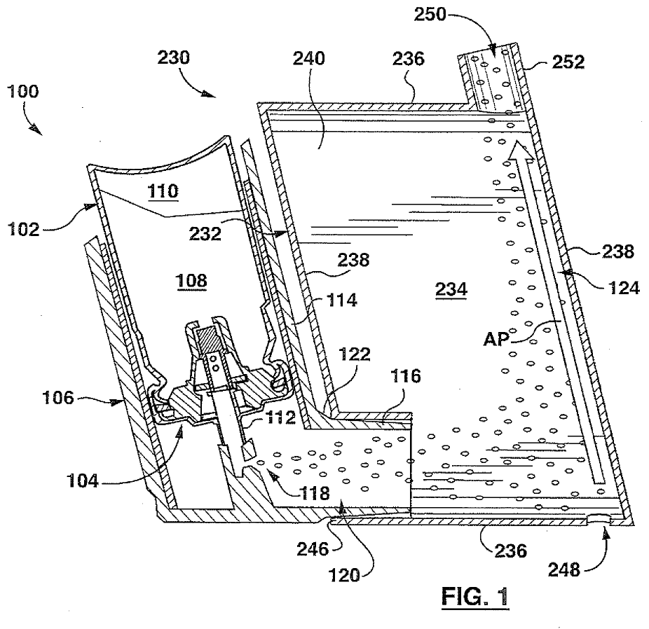

A metered-dose inhaler with an exemplary dilution spacer as shown in FIG. 1 may be used to administer the present nicotine formulation. The metered-dose inhaler 100 comprises a canister 102, a metering valve 104 and an actuator 106, with the canister 102 received in the body 114 of the actuator 106 such that the valve stem 112 of the metering valve 106, which is sealed to the canister 102, is received by the actuator nozzle 118, which is configured to generate a plume 120 from the formulation 108 in the canister 102. The actuator inlet 242 is configured to securely releasably interengage the actuator mouthpiece 116, and the metered-dose inhaler 100 is coupled to the dilution spacer 230 by interengagement of the actuator mouthpiece 116 and the actuator inlet 242, with the body 114 of the actuator 104 substantially flush with the edge of the enclosure 232. In this position, the actuator nozzle 118 will direct the plume 120 into the dilution chamber 234 through the actuator mouthpiece 116 and the actuator inlet 242.

To use, a user seals his or her mouth around the outlet 250 (e.g. seal his or her lips around the dilution spacer mouthpiece 252) and inhales through his or her mouth. This inhalation creates suction through the outlet 250 from outside the enclosure 232 which, because the ambient air inlet 248 is positioned opposite the outlet 250, draws ambient air into the enclosure 232 through the ambient air inlet 248 to generate an airflow path, denoted by arrow AP, from the ambient air inlet 248 through the dilution chamber 234 and out of the outlet 250 into the user's mouth. While inhaling during the early portion of the inhalation cycle, the user would push the canister 102 toward the actuator nozzle 118 so as to move the valve stem 112 (relative to the canister 102) into the dispensing position and release the metered quantity of medication 108 into the actuator nozzle 118 to generate the plume 120, as shown in FIG. 1. The actuator inlet 242 is positioned, relative to the ambient air inlet 248 and the outlet 250 so that when the plume 120 enters the dilution chamber 234 through the actuator inlet 242, the plume 120 has maximum distance within the confines of portability to develop, disperse and decelerate before it intersects the airflow path AP. As a result, airflow (generated by the user's inhalation) along the airflow path AP entrains and redirects the most deeply inhalable portion 124 of the plume 120 toward the outlet 250 so that it will be inhaled by the user. The portion of the plume 120 of larger particle size, that can penetrate the air current and would otherwise land in the user's mouth and throat in the absence of the dilution chamber 234, deposits onto the inside wall of the dilution chamber 234.

1. An orally inhalable formulation comprising nicotine or a pharmaceutically acceptable salt thereof, a monohydric alcohol, at least one menthol-containing essential oil, and a propellant, wherein the ratio of monohydric alcohol:nicotine by weight is no more than about 2.5:1, and the menthol-containing essential oil:nicotine ratio is about 0.25-2:1 w/w.

2. The formulation as defined in claim 1, wherein the monohydric alcohol is ethanol.

3. The formulation as defined in claim 1, wherein the menthol-containing essential oil:nicotine ratio is between 0.5-1.5:1 by weight.

4. The formulation according to claim 1, wherein the propellant is a hydrofluoroalkane selected from the group consisting of HFA 334a, 134a, 152a and 227ea.

5. The formulation according to claim 1, wherein the propellant is hydrofluoroolefin.

6. The formulation according to claim 1, wherein the ratio of monohydric alcohol:nicotine by weight is less than 2:1.

7. The formulation according to claim 1, wherein the formulation comprises nicotine and lactic acid at about a molar equivalent level.

8. An orally inhalable nicotine formulation comprising nicotine, or a pharmaceutically acceptable salt thereof, a monohydric alcohol in an amount of 6-12% by wt of the formulation, organic acid in an amount that results in a physiologic to a basic pH which is physiologically acceptable, menthol in an amount of 0.5-2:1 nicotine by wt and a propellant.

9. The nicotine formulation of claim 8, wherein the ratio of menthol to nicotine is 1-1.5:1 w/w.

10. The nicotine formulation of claim 8, wherein organic acid is added in an amount that results in a pH in the range of about 7.4-7.8.

11. The nicotine formulation of claim 8, wherein the organic acid is added in an amount of 0.4-0.6:1 acid:nicotine w/w.

12. The nicotine formulation of claim 8, wherein the organic acid is added in an amount of 0.5:1 acid:nicotine w/w.

13. The nicotine formulation of claim 8, wherein the organic acid is lactic acid.

14. The nicotine formulation of claim 8, wherein the monohydric alcohol in the formulation is about 9-10% by wt.

15. The nicotine formulation of claim 8, wherein the monohydric alcohol is ethanol.

16. The nicotine formulation of claim 8, comprising ethanol in an amount of 9-10% by wt of the formulation, lactic acid in an amount of 0.4-0.6:1 acid:nicotine w/w, menthol in an amount of 1-1.5:1 nicotine by wt and a hydrofluoroalkane propellant selected from the group consisting of HFA 334a, 134a, 152a and 227ea.

17. The nicotine formulation of claim 8, comprising nicotine. in amount of 0.125-2% by wt of the formulation.

18. A pressurized metered dose inhaler comprising an orally inhalable formulation as defined in claim 1.

19. A pressurized metered dose inhaler comprising an orally inhalable formulation as defined in claim 8.

20. The inhaler according to claim 19, comprising:
a pressurized canister, wherein the internal surface of the pressurized canister is treated with a plasma coating;
an actuator nozzle having a diameter of between 0.2 mm and 0.4 mm; and/or
a canister housed within an actuator which is coupled to a compact dilution spacer such that upon actuation an aerosol plume expands and decelerates within the dilution spacer before being redirected and leaving the spacer via a mouthpiece by force of inhalation.
